(19) United States
(12) Patent Application Publication
(10) Pub. No.: US 2023/0035300 A1
(43) Pub. Date: Feb. 2, 2023

(54) Method for detecting viruses

(71) Applicant: National Cheng Kung University, Tainan City (TW)

(72) Inventors: Jiunn-Der LIAO, Tainan City (TW); Han LEE, Tainan City (TW)

(21) Appl. No.: 17/386,600

(22) Filed: Jul. 28, 2021

(51) Int. Cl. G01N 21/65; G01N 33/569

(52) U.S. Cl. CPC G01N 21/658; G01N 33/56983

(57) ABSTRACT

The method of the present disclosure comprises the following steps: providing a SERS-active substrate and a Raman spectra virus database; applying a virus sample onto the SERS-active substrate; applying an incident light by a Raman spectrometer onto the SERS-active substrate to generate a Raman spectrum of the virus sample; and comparing the Raman spectrum of the virus sample with a Raman spectra virus database to identify the species of the virus sample. Herein, the SERS-active substrate comprises: a support; a dielectric layer disposed on the support, wherein a plurality of cavities are formed on a surface of the dielectric layer; and a plurality of noble metal clusters formed in the plurality of cavities.

### BACKGROUND

### 1. Field

The present disclosure relates to a method for detecting virus. More specifically, the present disclosure relates to a method for detecting viruses using the surface-enhanced Raman scattering (SERS) technology.

### 2. Description of Related Art

Rapid and accurate detection and identification of a dangerous pathogen is extremely helpful for a diagnosis or therapy of an early-stage disease caused by the pathogen. Currently, antibody-based bioarrays can be employed to monitor viruses and such techniques are, for example, enzyme-linked immunosorbent assays (ELISA), fluorescent antibody arrays, and serological testing. However, these techniques may meet some clinical problem such as being time-consuming, false positive, etc.

In recent years, as polymerase chain reaction (PCR) keeps advancing, related methods such as single-nucleotide polymorphism (SNP) in which a target DNA sequence is also proliferated, become alternative approaches to virus identification. However, complex purification and isolation steps of the target DNA are required to be performed prior to ELISA or PCR. Therefore, if such complex purification and isolation steps can be eliminated, identification of a target virus can be simplified and accelerated.

At present, in virus identification or detection, the major difficulties are associated with the complex purification and isolation, the detectable amount, apparent size, and dimension of virus, and the requirement of chemical identification databases for specific viruses. In general, rapid-screening detection is difficult to achieve at an early stage of virus infection since the virus amount often is unable to reach the detectable amount, more than 10⁶ plaque-forming units (PFU)/ml, in biomolecular assays where the purification procedure is employed. The reagents used in common biomolecular assays are far smaller than an apparent size and dimension of a virus. Thus, only a portion of the integral virus can be detected, resulting in incomplete virus information. Also, it is difficult to establish chemical identification information for the integral virus in a database.

Biomolecules or their fragments can be investigated by resonating mechanical cantilevers, evanescent wave biosensors, or atomic force microscopy, but these approaches are used only to measure the amount of the biomolecules. Alternatively, a labeling method can be employed to detect microorganisms by a particular functional group. However, it is difficult to prepare samples without contamination, or easy to overlap between signals of the labeling functional group and the target microorganism. Hence, these techniques consume a lot of time for detection and cannot meet a clinical requirement for rapid detection.

### SUMMARY

The object of the present disclosure is to provide a method for detecting virus using the surface-enhanced Raman scattering (SERS) technology.

The method of the present disclosure is accomplished by using a SERS-active substrate, which comprises: a support; a dielectric layer disposed on the support, wherein a plurality of cavities are formed on a surface of the dielectric layer; and a plurality of noble metal clusters formed in the plurality of cavities. The method of the present disclosure comprises the following steps: providing the aforesaid SERS-active substrate and a Raman spectra virus database; applying a virus sample onto the plurality of cavities of the SERS-active substrate; applying an incident light by a Raman spectrometer onto the plurality of noble metal clusters of the SERS-active substrate to generate a Raman spectrum of the virus sample; and comparing the Raman spectrum of the virus sample with a Raman spectra virus database to identify the species of the virus sample.

In the method of the present disclosure, the SERS-active substrate comprises the noble metal clusters formed in the cavities, and in particular, the noble metal clusters are formed in each of the cavities. When the SERS-active substrate of the present disclosure is used with a Raman spectrometer to detect the virus sample, the overall hot spots of each of the cavities are the collective results of small hot spots (from the noble metal clusters) created by the noble metal clusters. Thus, the intensity of the Raman signal of the virus sample can be enhanced by the hot spots, and it is possible to detect viruses accommodated by the cavities with the hot spots.

In the present disclosure, the material of the support is not particularly limited, and may include, for example, quartz, glass, silicon wafer, sapphire, polycarbonate (PC), polyimide (PI), polypropylene (PP), polyethylene terephthalate (PET) or other plastic or polymer material, or a combination thereof, but the present disclosure is not limited thereto.

In the present disclosure, the material of the dielectric layer may be a ceramic material. For example, the material of the dielectric layer may be a high-k ceramic material having a dielectric constant (k) ranging from 3.9 to 30. Specific examples of the high-k ceramic material include ZrO₂, TiO₂, HfO₂, Al₂O₃ or a combination thereof, but the present disclosure is not limited thereto.

In the present disclosure, the cavities formed on a surface of the dielectric layer may be arranged in an array. For example, the cavities may be arranged in an n x m array, wherein n and m are respectively an integral of 1 or more.

In the present disclosure, each of the plurality of cavities may have a bowl-like shape.

In the present disclosure, each of the plurality of cavities may have a depth ranging from 20 nm to 300 nm, for example, 20 nm to 280 nm, 20 nm to 250 nm, 20 nm to 230 nm, 20 nm to 200 nm, 20 nm to 180 nm, 20 nm to 150 nm, 25 nm to 150 nm, 25 nm to 130 nm, 30 nm to 130 nm, 30 nm to 100 nm or 40 nm to 100 nm. Herein, the depth of the cavities can be selected according to the size and dimension of the target viruses to be detected. In addition, the depth of the cavities can be the maximum depth of the cavities.

In the present disclosure, each of the plurality of cavities may have a width ranging from 100 nm to 1400 nm, for example, 100 nm to 1300 nm, 100 nm to 1200 nm, 100 nm to 1100 nm, 100 nm to 1000 nm, 100 nm to 900 nm, 100 nm to 800 nm, 100 nm to 700 nm, 100 nm to 600 nm, 100 nm to 500 nm, 150 nm to 500 nm, 150 nm to 400 nm, 200 nm to 400 nm or 200 nm to 300 nm. Herein, the width of each of the cavities may be selected according to the size and dimension of the target viruses to be detected. In addition, the width of the cavities can be the maximum width of the cavities.

In the present disclosure, the metal of the plurality of noble metal clusters may be Au, Ag or an alloy thereof. In one embodiment of the present disclosure, the metal may be Au.

In the present disclosure, each of the plurality of noble metal clusters has a disk-like shape with a thickness of several nanometers. The disk-like shape can be a flat shape. The outline of the disk-like shape is not particularly limited, and can be, for example, circular or irregular. In addition, each of the plurality of noble metal cluster may have a width ranging from 0.5 nm to 50 nm, for example, 0.5 nm to 45 nm, 0 5 nm to 40 nm, 0.5 nm to 35 nm, 0.5 nm to 30 nm, 1 nm to 30 nm, 1 nm to 25 nm, 2 nm to 25 nm, 2 nm to 20 nm, 3 nm to 20 nm, 3 nm to 15 nm, 4 nm to 15 nm, 4 nm to 10 nm or 5 nm to 10 nm. Herein, the width of the noble metal cluster refers to the average width of the disk-like shape of the noble metal clusters, which may be chosen according to the size and dimension of the targeted viruses to be detected. Moreover, the average thickness of the disk-like shape of the noble metal clusters may be ranged from 0.5 nm to 5 nm. In addition, a space between two adjacent metal clusters may be in a range from 5 nm to 10 nm, and this space can be referred to the average minimum distance between two adjacent metal clusters.

In the present disclosure, each of the cavities may be accommodating 0˜3 viruses according to the target viruses (for example, the size of the virus particle) to be detected. For example, one cavity may accommodate 1, 2 or 3 viruses, but another cavity is empty (i.e. 0 virus) when the virus sample is applied onto the SERS-active substrate. How many viruses are accommodated into the cavity may, for example, depend upon the amount of the viruses in the virus sample.

In the present disclosure, the distance between two adjacent cavities of the plurality of cavities may be in a range from 10 nm to 200 nm, for example, 10 nm to 190 nm, 10 nm to 180 nm, 10 nm to 170 nm, 10 nm to 160 nm, 10 nm to 150 nm, 10 nm to 140 nm, 10 nm to 130 nm, 10 nm to 120 nm, 10 nm to 110 nm, 10 nm to 100 nm, 15 nm to 100 nm, 15 nm to 90 nm, 20 nm to 90 nm, 20 nm to 80 nm, 25 nm to 80 nm, 25 nm to 70 nm or 30 nm to 70 nm; but the present disclosure is not limited thereto. Herein, the distance between two adjacent cavities can be the minimum distance between two adjacent cavities.

In the present disclosure, the wavelength of the incident light provided by the Raman spectrometer can be adjusted according to the size (for example, the depth) of the cavities, or the viruses (for example, the size and the kind of the virus particle) to be detected. Thus, the optimized signal of the SERS effect can be obtained. In addition, the power of the incident light provided by the Raman spectrometer can be in a range from 0.3 mW to 40 mW, for example, 0.3 mW to 30 mW, 0.3 mW to 20 mW, 0.5 mW to 20 mW, 0.5 mW to 15 mW, 1 mW to 15 mW, 1 mW to 10 mW, 1.5 mW to 10 mW, 1.5 mW to 5 mW, 1.5 mW to 4.5 mW, 2 mW to 4.5 mW, 2.5 mW to 4.5 mW, 2.5 mW to 4 mW or 3 mW to 4 mW. If the power is too large, the virus sample may be degraded.

In the present disclosure, the species of the virus to be detected is not particularly limited. For example, the virus may be virus with spike protein, such as SARS-CoV-2 virus or its variations.

It is known that the conventional detection method such as nucleic acid tests (for example, polymerase chain reaction (PCR) or reverse transcription PCR (RT-PCR)) or serological tests (for example, enzyme-linked immunosorbent assay (ELISA) or lateral flow immunoassay (LFIA)) is time-consuming or has its shortage. However, once the virus becomes a pandemic, a rapid and convincing detection system or method is required to timely identify the types of the virus in the patients, and thus the spread of the disease can be effectively controlled. Herein, the method of the present disclosure is performed by the SERS technology, so the species of the viruses can be identified rapidly and accurately. In addition, because the SERS effect can be improved by the noble metal clusters in the cavities in the SERS-active substrate of the present disclosure, the species of the viruses still can be identified even though the amount of the viruses in the virus sample is low.

Other novel features of the disclosure will become more apparent from the following detailed description when taken in conjunction with the accompanying drawings.

### BRIEF DESCRIPTION OF THE DRAWINGS

FIG. 1A to FIG. 1D are cross-sectional views showing the preparation of a SERS-active substrate according to Embodiment 1 of the present disclosure.

FIG. 2 is a perspective view showing a support and a dielectric layer of a SERS-active substrate according to Embodiment 1 of the present disclosure.

FIG. 3 is an enlarge view of a cavity of a SERS-active substrate according to Embodiment 1 of the present disclosure.

FIG. 4A to FIG. 4D are top view SEM images of SERS-active substrates according to Embodiment 1 of the present disclosure.

FIG. 5A is SERS spectra of SARS-CoV-2 S pseudovirus taken at the same position of the array for 5 times.

FIG. 5B is a diagram showing the intensities of the SERS spectra of SARS-CoV-2 S pseudovirus taken at the same position of the array for 5 times.

FIG. 6A is SERS spectra of SARS-CoV-2 S pseudovirus taken at 6 different locations of the array.

FIG. 6B is a diagram showing the intensities of the SERS spectra of SARS-CoV-2 S pseudovirus taken at 6 different locations of the array.

FIG. 7 is SERS spectra of SARS-CoV-2 S pseudovirus versus VSV-G pseudovirus.

FIG. 8 is SERS spectra of SARS-CoV-2 S pseudovirus versus H1N1.

FIG. 9 is SERS spectra of SARS-CoV-2 S pseudovirus versus H3N2.

FIG. 10 is a top view of a SERS-active substrate according to Embodiment 2 of the present disclosure.

### DETAILED DESCRIPTION OF EMBODIMENT

Different embodiments of the present invention are provided in the following description. These embodiments are meant to explain the technical content of the present invention, but not meant to limit the scope of the present invention. A feature described in an embodiment may be applied to other embodiments by suitable modification, substitution, combination, or separation.

It should be noted that, in the present specification, when a component is described to have an element, it means that the component may have one or more of the elements, and it does not mean that the component has only one of the element, except otherwise specified.

Moreover, in the present specification, the ordinal numbers, such as “first” or “second”, are used to distinguish a plurality of elements having the same name, and it does not means that there is essentially a level, a rank, an executing order, or an manufacturing order among the elements, except otherwise specified. A “first” element and a “second” element may exist together in the same component, or alternatively, they may exist in different components, respectively. The existence of an element described by a greater ordinal number does not essentially means the existent of another element described by a smaller ordinal number.

Moreover, in the present specification, the terms, such as “top”, “bottom”, “left”, “right”, “front”, “back”, or “middle”, as well as the terms, such as “on”, “above”, “under”, “below”, or “between”, are used to describe the relative positions among a plurality of elements, and the described relative positions may be interpreted to include their translation, rotation, or reflection.

Moreover, in the present specification, when an element is described to be arranged “on” another element, it does not essentially means that the elements contact the other element, except otherwise specified. Such interpretation is applied to other cases similar to the case of “on”.

Moreover, in the present specification, the terms, such as “preferably” or “advantageously”, are used to describe an optional or additional element or feature, and in other words, the element or the feature is not an essential element, and may be ignored in some embodiments.

Moreover, in the present specification, when an element is described to be “suitable for” or “adapted to” another element, the other element is an example or a reference helpful in imagination of properties or applications of the element, and the other element is not to be considered to form a part of a claimed subject matter; similarly, except otherwise specified; similarly, in the present specification, when an element is described to be “suitable for” or “adapted to” a configuration or an action, the description is made to focus on properties or applications of the element, and it does not essentially mean that the configuration has been set or the action has been performed, except otherwise specified.

Moreover, in the present specification, a value may be interpreted to cover a range within ±10% of the value, and in particular, a range within ±5% of the value, except otherwise specified; a range may be interpreted to be composed of a plurality of subranges defined by a smaller endpoint, a smaller quartile, a median, a greater quartile, and a greater endpoint, except otherwise specified.

### Embodiment 1—Preparation of the SERS-active Substrate

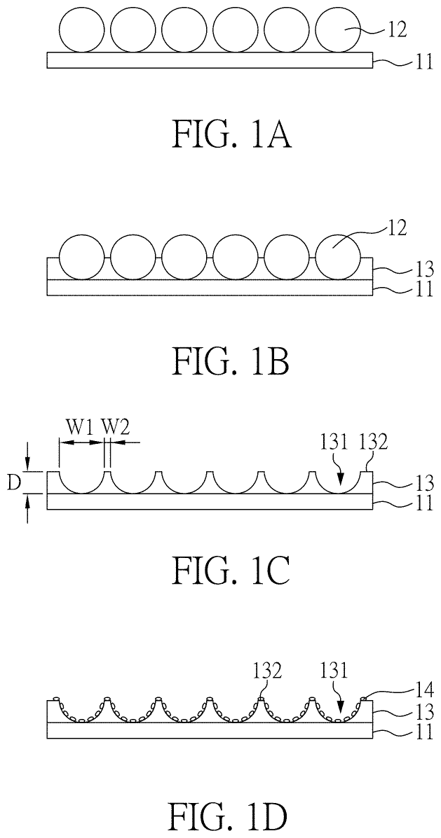

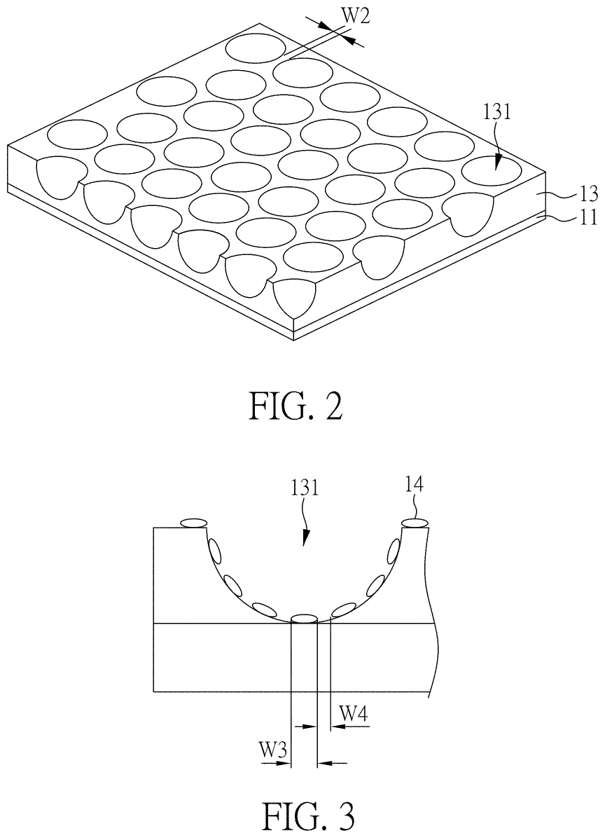

FIG. 1A to FIG. 1D are cross-sectional views showing the preparation of a SERS-active substrate according to the present embodiment. FIG. 2 is a perspective view showing a support and a dielectric layer of a SERS-active substrate according the present embodiment. FIG. 3 is an enlarge view of a cavity of a SERS-active substrate according to the present embodiment.

As shown in FIG. 1A, a support 11 was provided, wherein the support 11 was a silicon piece support. After cleaning the support 11, a plurality of nanoparticles 12 were formed on the support 11. In the present embodiment, the nanoparticles 12 were polystyrene (PS) nanoparticles, but the present disclosure is not limited thereto. In another embodiment of the present disclosure, the nanoparticles 12 may be polyvinyl alcohol (PVA) nanoparticles, polyethylene glycol (PEG) nanoparticles or other polymer nanoparticles. In addition, in the present embodiment, the nanoparticles 12 with three different diameters (150 nm, 250 nm, and 350 nm) were used to prepare three different SERS-active substrates.

After slightly annealing at 50° C. for 10 min, a precursor solution of ZrO₂ was applied on the support 11 by a spin-coating process. The precursor solution of ZrO₂ was prepared with a mixture of zirconium tetrachloride (ZrCl₄, 98%, Acros Organics, Pittsburgh, Pa., USA) and isopropanol (99.8%, Panreac Applichem, Darmstadt, Germany), and the precursor solution of ZrO₂ was left to stand for ˜24 hr until it achieved a gel-like consistency before use for spin-coating. Then, the support 11 was heated (heating rate: 5° C./min), and kept at 600° C. for 3 hr to perform the annealing process. As shown in FIG. 1B, after cooling down to room temperature, a dielectric layer 13 formed by ZrO₂ was obtained.

As shown in FIG. 1C and FIG. 2, after removing the nanoparticles 12, the cavities 131 having the shapes corresponding to the nanoparticles 12 were formed. Then, as shown in FIG. 1D, Au atoms were deposited onto the dielectric layer 13 and formed as clusters with the use of an electron beam evaporator (VT1-10CE, ULVAC), wherein the pressure of the chamber was about 7×10⁻⁶ ton. The deposition rate was 0.1 Å/sec until the value achieved 50 Å, and then the deposition rate was changed to 1 Å/sec until the instrument parameter achieved 3 nm. The obtained width the disk-like cluster was 30˜60 nm with a flatten thickness of several nm and a spacing between/among adjacent clusters in the range of 5˜10 nm.

After the aforesaid process, the SERS-active substrate of the present embodiment is accomplished. As shown in FIG. 1D, the SERS-active substrate comprises: a support 11; a dielectric layer 13 disposed on the support 11, wherein a plurality of cavities 131 are formed on a surface 132 of the dielectric layer 13; and a plurality of noble metal clusters 14 formed in the cavities 131. Herein, the dielectric layer 13 is a ZrO₂ layer, and the noble metal clusters are Au clusters.

As shown in FIG. 1C and FIG. 2, the cavities 131 are arranged in a hexagonal array, and each of the cavities 131 has a bowl-like shape. In the SERS-active substrate prepared by using the nanoparticles 12 with the diameter of 250 nm, the obtained cavities 131 have the depth D (the maximum depth) of 60 nm to 80 nm and the width W1 (the maximum width) of 240 nm to 250 nm. In addition, the distance W2 (the minimum distance) between two adjacent cavities 131 is 50 nm. The aforesaid features can be confirmed by SEM (scanning electron microscope) and AFM (atomic force microscope) images as well as the cross-sectional profiling of the SERS-active substrate, but the present disclosure is not limited thereto.

Furthermore, as shown in FIG. 3, the noble metal clusters 14 have the disk-like shapes, the width W3 of noble metal clusters 14 was estimated 30˜60 nm, and the noble metal clusters 14 have the average spacing distance W4 of 5˜10 nm The aforesaid features can be confirmed by AFM images of the SERS-active substrate, but the present disclosure is not limited thereto.

The depth D, the width W1 and the distance W2 of the cavities, the width W3 of the noble metal clusters 14 and the spacing W4 between adjacent noble metal clusters 14 may be adjusted according to the need, and the present disclosure is not limited to the aforesaid values.

In the present embodiment, high-resolution thermal field emission scanning electron microscope coupled with energy dispersive X-Ray spectroscopy (FE-SEM/EDS, JSM-7000, JEOL, Tokyo) and atomic force microscope (AFM, Dimension Icon, Bruker, Karlsruhe, Germany) can be utilized to analyze the morphology and composition of the obtained SERS-active substrate. Images of the SERS-active substrate were taken at secondary electron imaging mode at an accelerating voltage of 10 kV and a current of 8×10⁻⁸ Å. In addition, the width of the noble metal clusters and the spacing between the noble metal clusters can be measured by AFM (Dimension Icon, Bruker, Karlsruhe, Germany). The PF-QNM of AFM was used to obtain the image of the SERS-active substrate, wherein the scan rate was 0.3 Hz and the scan line was 256. However, the present disclosure is not limited thereto, and any know method can be used to analyze the morphology and composition of the obtained SERS-active substrate.

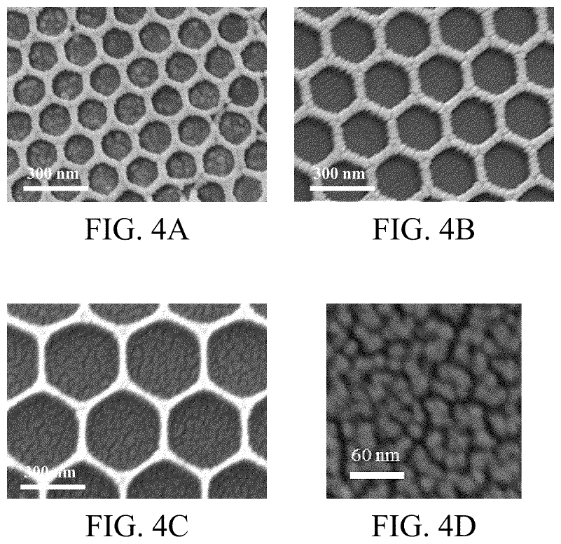

FIG. 4A to FIG. 4D are top view SEM images of SERS-active substrates of the present embodiment, wherein FIG. 4A to FIG. 4C are top view SEM images of SERS-active substrates respectively prepared by nanoparticles having diameters of 150 nm, 250 nm and 350 nm, and FIG. 4D is an enlarge top view SEM image of a cavity of the obtained SERS-active substrate. From the result shown in FIG. 4, it can be found that the width of noble metal clusters 14 was estimated 30˜60 nm, and the noble metal clusters 14 have the average spacing distance of 5˜10 nm.

Note that one Raman spot may cover a few cavities. For example, when one Raman spot has a diameter of 1 μm, one Raman spot may cover about 11 cavities of the SERS-active substrate prepared by nanoparticles having diameter of 250 nm, and each of the cavities covered by the Raman spot may or may not contain the virus particles.

### Embodiment 2—Detection System

The SERS-active substrate of Embodiment 1 may be used with a Raman spectrometer to form a detection system. Herein, the Raman spectrometer can provide an incident laser onto the noble metal clusters of the SERS-active substrate to obtain a Raman scattering signal, and then output a Raman spectrum.

### Testing Example

The SERS-active substrate of Embodiment 1 and the detection system of Embodiment 2 is used in the present texting example. The procedure for detecting a virus comprises the following steps: providing a SERS-active substrate and a Raman spectra virus database; applying a virus sample onto the plurality of cavities of the SERS-active substrate; applying an incident light by a Raman spectrometer onto the noble metal clusters of the SERS-active substrate to generate a Raman spectrum of the virus sample; and comparing the Raman spectrum of the virus sample with a Raman spectra virus database to identify the species of the virus sample.

### Preparation of SARS-CoV-2 S Pseudovirus

SARS-CoV-2 S pseudovirus was produced following the report of Huang et al. with minor modifications (Huang, S. W., Tai, C. H., Hsu, Y. M., Cheng, D., Hung, S. J., Chai, K. M., Wang, Y. F., Wang, J. R., 2020. Assessing the application of a pseudovirus system for emerging SARS-CoV-2 and re-emerging avian influenza virus H5 subtypes in vaccine development. Biomed. J. 43, 375-387). The lentiviral vector system was provided by the National RNAi Core of Academia Sinica Taiwan. De novo synthesis was performed to obtain sequences of the spike protein, which were then cloned into the pMD.G plasmid to express SARS-CoV-2 S pseudoviruses. Cells were transfected with a total of 40 μg pCMVdeltaR8.91, pLAS3w and pMD.G (VSV-G pseudo-type lentivirus, or VSV-G pseudovirus) or pcDNA3.1-spike Wuhan plasmids (SARS-CoV-2 S pseudovirus).

### Preparation of H1N1 virus and H3N2 Virus

The samples containing H1N1 or H3N2 virus used in the present embodiment was provided by Dr. Wang, Jen-Ren in Department of Medical Laboratory Science and Biotechnology, College of Medicine, National Cheng Kung University, and the virus stains were obtained from Department of Pathology, National Cheng Kung University Hospital. MOCK cells (Madin-Darby Canine Kidney cells) were cultured in 75T culture flasks. After a full monolayer of cells was obtained, the original medium was removed, and the cells were washed with PBS buffer twice. After removing the PBS buffer, the virus fluid was placed into a 37° C. water bath to thaw quickly. To infect the cells, the thawed cells were added into a virus culture tube with a single cell layer, followed by shaking evenly, so that the cell surfaces can completely contact with the virus liquid. Then, the culture was placed into an incubator at 35° C. for 1 hour. After adding suitable amount of the culture containing influenza virus, the obtained culture was again placed into the incubator at 35° C. When 75% of the cells had the cytopathic effect (CPE), the viruses were divided into portions and stored.

### Detection

The SERS experiments were performed with the use of the Raman spectrometer (Confocal Micro and Nano Raman Spectrometer, UniDRON, CL Technology Co. Ltd.) with a laser source of 633 nm and a maximum laser power of 35 mW. The pseudovirus sample was dropped onto the SERS-active substrate and then covered with a glass cover slip before subjecting to the Raman laser for measurement. In this experiment, spectra were taken 5 times on the same spot of the detection to investigate the effect of exposing the same area on the change of peak intensities, and 6 different locations on the same SERS-active substrate were tested to see the reproducibility of results and consistency of the SERS spectra. In addition, the laser power of 3.5 mW was used for measurement.

### Result

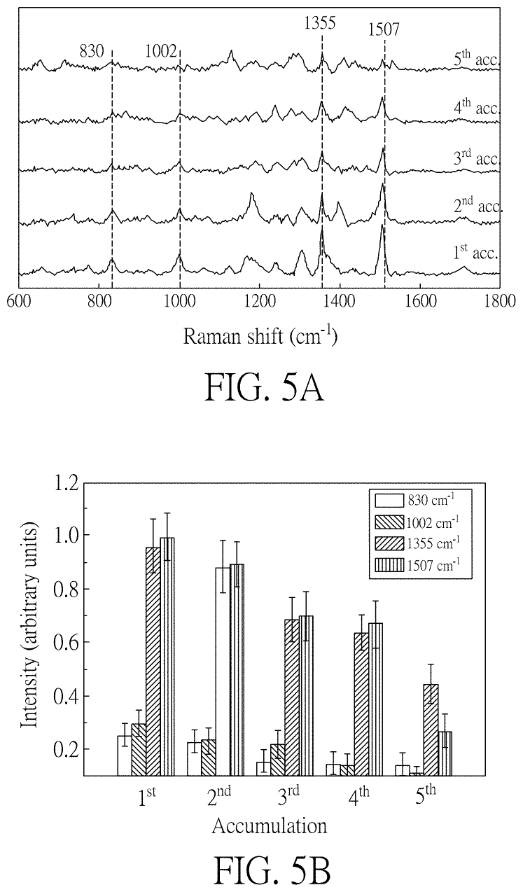

FIG. 5A is SERS spectra of SARS-CoV-2 S pseudovirus taken at the same position of the array for 5 times, and FIG. 5B is a diagram showing the intensities of the SERS spectra of SARS-CoV-2 S pseudovirus taken at the same position of the array for 5 times. These results indicate that the same characteristic peaks 830, 1002, 1355, and 1507 cm⁻1 are found at the same Raman shifts throughout the 5 measurements but with a decline in intensities with the increasing use of the same spot (from 1^{st} to 5^{th} accumulation). This decline in intensities is due to the damage caused by the laser to both the array and the analytes by the continuous use of the same spot.

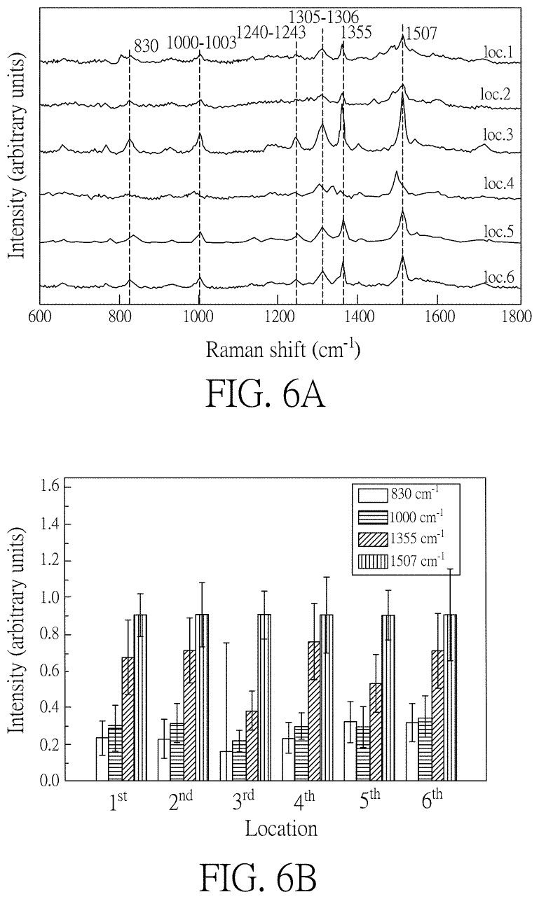

FIG. 6A is SERS spectra of SARS-CoV-2 S pseudovirus taken at 6 different locations of the array, and FIG. 6B is a diagram showing the intensities of the SERS spectra of SARS-CoV-2 S pseudovirus taken at 6 different locations of the array. These results indicate that characteristic peaks as marked FIG. 6A are strong regardless of location, but it should be noted that some peaks are not as apparent in some locations. This is most likely due to the non-uniformity of microstructures in the array.

From the results shown in FIG. 5A to FIG. 6B, four characteristic peaks about 830, 1000-1003, 1355, and 1507 cm⁻¹ are found at the same Raman shifts in the SERS spectra of SARS-CoV-2 S pseudovirus.

As shown in FIG. 1C, in the SERS-active substrate used herein, each of the cavities 131 has a bowl-like shape, the obtained cavities 131 have the depth D (the maximum depth) of 60 nm to 80 nm and the width W1 (the maximum width) of 240 nm to 250 nm. Thus, one cavity 131 could hold up to 3 SARS-CoV-2 S pseudoviruses with the virus particle size of about 100 nm. In addition, when the virus particles are located in the cavities 131, the overall hot spots of the cavities 131 depend on the collective result of small hot spots created by each Au clusters. In addition, ZrO₂ of the dielectric layer 13 also contributes a chemical enhancement effect.

The aforesaid results indicate that the SERS-active substrate of the present disclosure indeed can distinguish live SARS-CoV-2 S pseudovirus using the surface-enhanced Raman scattering (SERS) technology.

FIG. 7 is SERS spectra of SARS-CoV-2 S pseudovirus versus VSV-G pseudovirus, wherein a comparison to detect SARS-CoV-2 with VSV-G pseudoviruses using the two SERS-active substrates. Since the components of VSV-G pseudovirus is known as it is widely studied, it could serve as a reference to SARS-CoV-2 S pseudovirus to determine the peaks that belongs to SARS-CoV-2 S pseudovirus. The SERS spectra of both pseudoviruses are shown in the figure as well as the corresponding subtracted spectra. The subtracted spectra represented the subtraction of the VSV-G pseudovirus spectra from the SARS-CoV-2 S pseudovirus spectra; the positive peaks on the subtracted spectra then correspond to peaks of SARS-CoV-2 S pseudovirus, while the negative peaks are that of the VSV-G pseudovirus. The results show that the SERS-active substrate of the present embodiment is capable of detecting both pseudoviruses.

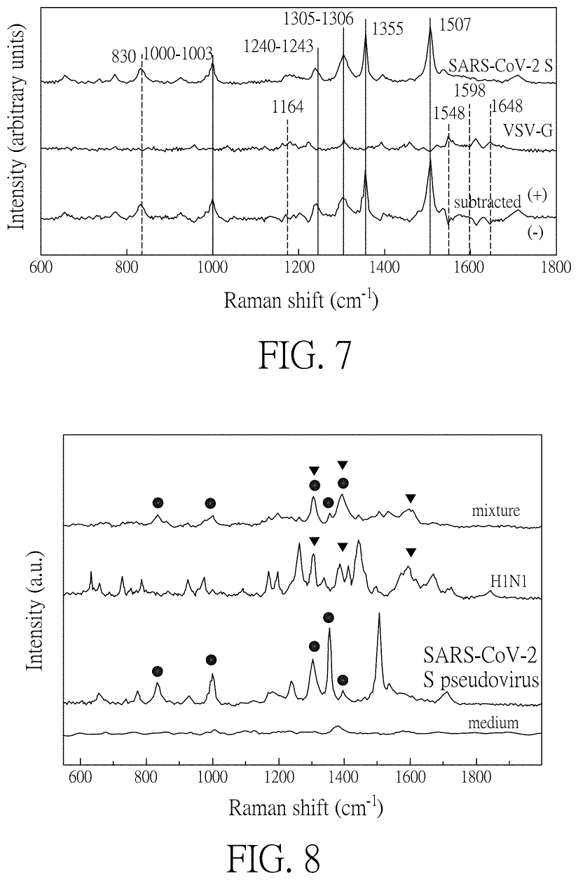

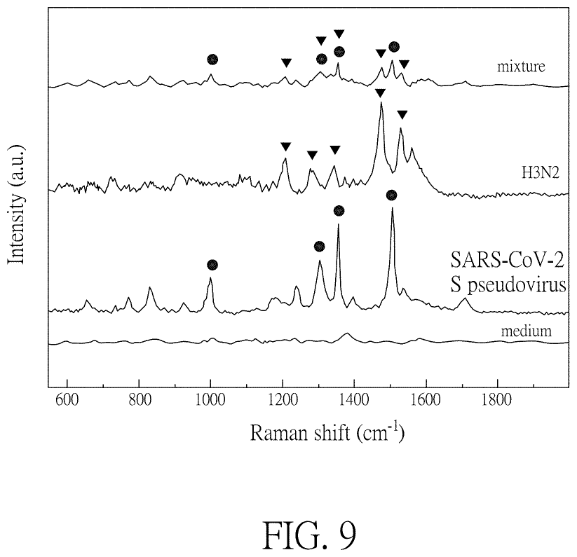

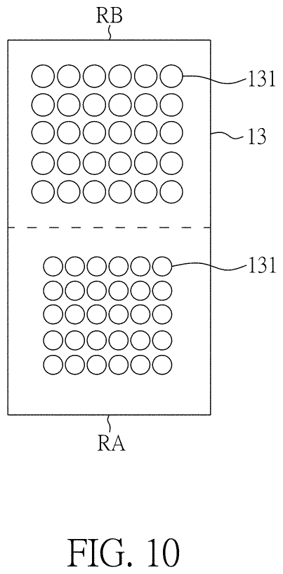

FIG. 8 is SERS spectra of SARS-CoV-2 pseudovirus, H1N1 and a mixture thereof, and FIG. 9 is SERS spectra of SARS-CoV-2 pseudovirus, H3N2 and a mixture thereof. As shown in FIG. 8 and FIG. 9, when a sample containing multiple viruses (SARS-CoV-2 pseudovirus and H1N1 in FIG. 8, and SARS-CoV-2 pseudovirus and H3N2 in FIG. 9), it is possible to detect the multiple viruses at the same time when using the SERS-active substrate of the present disclosure.

Embodiment 2—Preparation of SERS-active Substrate

FIG. 5 is a top view of a SERS-active substrate according to Embodiment 2 of the present disclosure and, in particular, the pattern of the dielectric layer of the SERS-active substrate.

The process for preparing the SERS-active substrate is similar to that shown in Embodiment 1, except that two different arrays are formed on the dielectric layer 13. Thus, the dielectric layer 13 of the present embodiment comprises two regions RA and RB. Herein, the widths of the cavities 131 in the region RA and the widths of the cavities 131 in the region RB are different, which can be accomplished by forming the dielectric layer 13 with the nanoparticles 12 having the different diameters. For example, during the step for forming the dielectric layer 13, the cavities 131 in the region RA may be formed by using the nanoparticles 12 with the diameter of 250 nm, and the cavities 131 in the region RB may be formed by using the nanoparticles 12 with the diameter of 350 nm; but the present disclosure is not limited thereto.

When a virus sample is detected, the intensities of the SERS spectra can be taken at plural locations of the array. When the characteristic peaks of the virus (for example, 830, 1000-1003, 1355, and 1507 cm⁻¹ for the SARS-CoV-2 S pseudovirus) to be detected are found, it means that the virus sample contains the target virus particles. In addition, by analyzing the percentage of the locations that the characteristic peaks of the virus are found, it is possible to know the relative amount of the virus particles contained in the virus sample.

In addition, when a virus sample is loaded into the SERS-active substrate, the virus particles can be entrapped into the cavities. When a Raman spectrometer is used to detect the virus sample with light having specific wavelength (for example, 633 nm or 785 nm), hot spots are generated by the noble metal clusters. Thus, the Raman signals of the virus particles can be enhanced.

Although the present disclosure has been explained in relation to its embodiment, it is to be understood that many other possible modifications and variations can be made without departing from the spirit and scope of the disclosure as hereinafter claimed.

1. A method for a detecting virus, comprising the following steps:
providing a SERS-active substrate and a Raman spectra virus database, wherein the SERS-active substrate comprises:
a support;
a dielectric layer disposed on the support, wherein a plurality of cavities are formed on a surface of the dielectric layer; and
a plurality of noble metal clusters formed in the plurality of cavities, applying a virus sample onto the plurality of cavities of the SERS-active substrate;
applying an incident light by a Raman spectrometer onto the plurality of noble metal clusters of the SERS-active substrate to generate a Raman spectrum of the virus sample; and
comparing the Raman spectrum of the virus sample with a Raman spectra virus database to identify the species of the virus sample,
wherein the virus has a spike protein.

2. The method of claim 1, wherein the plurality of cavities are arranged in an array.

3. The method of claim 1, wherein each of the plurality of cavities has a bowl-like shape.

4. The method of claim 1, wherein each of the plurality of cavities has a depth ranging from 20 nm to 300 nm.

5. The method of claim 4, wherein each of the plurality of cavities has the depth ranging from 40 nm to 100 nm.

6. The method of claim 1, wherein each of the plurality of cavities has a width ranging from 100 nm to 1400 nm.

7. The method of claim 6, wherein each of the plurality of cavities has the width ranging from 200 nm to 300 nm.

8. The method of claim 1, wherein a metal of the plurality of noble metal clusters is Au, Ag or an alloy thereof.

9. The method of claim 8, wherein the metal of the plurality of noble metal clusters is Au.

10. The method of claim 1, wherein each of the plurality of noble metal clusters has a disk-like shape.

11. The method of claim 1, wherein each of the plurality of noble metal clusters has a width ranging from 0.5 nm to 50 nm.

12. The method of claim 11, wherein each of the plurality of noble metal clusters has the width ranging from 5 nm to 10 nm.

13. The method of claim 1, wherein a spacing between two adjacent metal clusters of the plurality of metal clusters is in a range from 5 nm to 10 nm.

14. The method of claim 1, wherein a distance between two adjacent cavities of the plurality of cavities is in a range from 10 nm to 200 nm.

15. The method of claim 14, wherein the distance between two adjacent cavities of the plurality of cavities is in a range from 30 nm to 70 nm.

16. The method of claim 1, wherein the dielectric layer comprises a ceramic material with dielectric constant ranging from 3.9 to 30.

17. The method of claim 16, wherein the ceramic material is ZrO2, HfO2, Al2O3 or a combination thereof.

18. (canceled)

19. The method of claim 1, wherein the virus is SARS-CoV-2 virus or its variations.
